(19) United States
(12) Patent Application Publication
(10) Pub. No.: US 2024/0052387 A1
(43) Pub. Date: Feb. 15, 2024

(54) VARIANTS OF GALA REDUCTASE AND THEIR USES

(71) Applicants: JOHANN WOKFGANG GOETHE-UNIVERSITAT FRANKFURT AM MAIN, FRANKFURT AM MAIN (DE); ROSALIND FRANKLIN UNIVERSITY OF MEDICINE AND SCIENCE, NORTH CHICAGO, IL (US)

(72) Inventors: SIMON HARTH, FRANKFURT AM MAIN (DE); MISLAV OREB, FRANKFURT AM MAIN (DE); JUN-YONG CHOE, GREENVILLE, NC (US)

(21) Appl. No.: 17/766,552

(22) PCT Filed: Oct. 8, 2020
(86) PCT No.: PCT/EP2020/078295

§ 371 Date: Apr. 5, 2022

Related U.S. Application Data
(60) Provisional application No. 62/912,195, filed on Oct. 8, 2019.

(51) Int. Cl. C12P 17/06; C12P 7/58; C12N 9/04; C12N 15/81

(52) U.S. Cl. CPC C12P 17/06; C12P 7/58; C12N 9/0006; C12Y 101/01067; C12Y 101/01; C12N 15/81; C12N 2800/102

(57) ABSTRACT

The present invention relates to polypeptides which are galacturonate (GalA) reductase variants comprising at least one amino acid substitution at a position corresponding to K261 and/or R267. The present invention further relates to nucleic acid molecules encoding the polypeptides and to host cells containing said nucleic acid molecules. The present invention further relates to a method for the production of L-galactonate (GalOA) and/or other bio-based compounds, comprising the expression of said nucleic acid molecules, preferably in said host cells. The present invention also relates to the use of the polypeptides, nucleic acids molecule or host cells for the production of L-galactonate (GalOA) and/or other bio-based compounds, and/or for the recombinant fermentation of biomaterial containing D-galacturonate (GalA).

The present invention relates to polypeptides which are galacturonate (GalA) reductase variants comprising at least one amino acid substitution at a position corresponding to K261 and/or R267. The present invention further relates to nucleic acid molecules encoding the polypeptides and to host cells containing said nucleic acid molecules. The present invention further relates to a method for the production of L-galactonate (GalOA) and/or other bio-based compounds, comprising the expression of said nucleic acid molecules, preferably in said host cells. The present invention also relates to the use of the polypeptides, nucleic acids molecule or host cells for the production of L-galactonate (GalOA) and/or other bio-based compounds, and/or for the recombinant fermentation of biomaterial containing D-galacturonate (GalA).

### BACKGROUND OF THE INVENTION

D-galacturonate (GalA), among sugars such as glucose, galactose and arabinose, is one of the major components of pectin, with a mass content of approximately 20%, e.g. in sugar beet pulp.

The worldwide production of pectin-containing biomass is specified in millions of tons (Protzko et al., 2018; Schmitz et al., 2019). Pectin-containing biomass accrues in large amounts, such as during saccharose extraction from sugar beets or during production of fruit juices, in particular from citrus fruits. Besides its use as gelating agent or feedingstuff, pectin is as of yet not used in a biotechnological manner.

The enzymatic conversion of GalA as one of the major components of pectin into L-galactonate (GalOA) is of interest, because:
1) It can serve as the first step of the GalA catabolism in fungi, e.g. Trichoderma reesei, Aspergillus niger. Establishing this metabolic pathway in biotechnological relevant organisms such as Saccharomyces cerevisiae can be used for the biotechnological use of GalA as carbon source for the production of miscellaneous products.
2) GalOA is a potentially interesting compound itself, which belongs to the family of polyhydroxy acids. Related compounds, such as gluconate (E574), have a long history of industrial use with applications in different areas of cosmetics and food industry, e.g. as complexing agent or acidifier. In addition, GalOA can be converted into L-galactono-γ-lacton (GgL), which is an analog to glucono-δ-lactone (E575).
3) L-galactono-γ-lacton is the direct precursor for the biosynthesis of L-ascorbic acid (vitamin C), wherein only one further enzymatic step is necessary.

The enzymatic conversion of GalA into GalOA is carried out by GalA reductases (EC 1.1.1.365), which belong to the family of aldo/ketoreductases. All known GalA reductases are exclusively or preferably NADPH-dependent. In the most biotechnological relevant organisms such as Saccharomyces cerevisiae the availability of NADPH is limited, because this cofactor is necessary in reactions of anabolic pathways, whereas the non-phosphorylated form of the cofactor (NADH) is sufficiently provided by the catabolic pathway, mainly by glycolysis. Even though it would be possible to increase the provision of NADPH by metabolic engineering, the changes which would be necessary are complex and do not always result in the desired outcome. Thus it would be advantageous to develop NADH-dependent enzymes for the enzymatic conversion of GalA into GalOA.

Thus, there is a need in the art for improved means and methods for a biotechnological conversion of GalA into GalOA.

### SUMMARY OF THE INVENTION

According to the present invention this object is solved by a polypeptide comprising an amino acid substitution at a position corresponding to K261 and/or R267 of the amino acid sequence of SEQ ID NO: 1, wherein the polypeptide has at least 80%, preferably at least 81%, more preferably at least 90% or 95% sequence identity with the amino acid sequence of SEQ ID NO: 1.

According to the present invention this object is solved by a nucleic acid molecule, coding for a polypeptide according to the present invention.

According to the present invention this object is solved by a host cell, containing a nucleic acid molecule of the present invention and preferably expressing said nucleic acid molecule, wherein said host cell is preferably a fungus cell and more preferably a yeast cell.

According to the present invention this object is solved by a method for the production of L-galactonate (GalOA) and/or other bio-based compounds, comprising the expression of a nucleic acid molecule according to the present invention, preferably in a host cell according to the present invention.

According to the present invention this object is solved by using a polypeptide according to the present invention, a nucleic acid molecule according to the present invention, or a host cell according to the present invention for the production of L-galactonate (GalOA) and/or other bio-based compounds.

According to the present invention this object is solved by using a polypeptide according to the present invention, a nucleic acid molecule according to the present invention, or a host cell according to the present invention for the recombinant fermentation of biomaterial containing D-galacturonate (GalA), and/or for the recombinant fermentation of biomaterial containing D-galacturonate (GalA) and glucose and/or other neutral sugar(s), such as galactose, arabinose or xylose.

### DESCRIPTION OF THE PREFERRED EMBODIMENTS OF THE INVENTION

Before the present invention is described in more detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art. For the purpose of the present invention, all references cited herein are incorporated by reference in their entireties.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “0.1 to 20” should be interpreted to include not only the explicitly recited values of 0.1 to 20, but also include individual values and sub-ranges within the indicated range. Thus, included in this numerical range are individual values such as 0.1, 0.2, 0.3, 0.4, 0.5, 0.6, 0.7, 0.8, 0.9, 1, 1.1 . . . 19.6, 19.7, 19.8, 19.9, 20 and sub-ranges such as from 1 to 10, 0.5 to 5, etc. This same principle applies to ranges reciting only one numerical value, such as “at least 90%”. Furthermore, such an interpretation should apply regardless of the breadth of the range or the characteristics being described.

GalA Reductase Variants

As discussed above, the present invention provides galacturonate (GalA) reductase variants.

In particular, the present invention provides a polypeptide comprising an amino acid substitution at a position corresponding to K261 and/or R267 of the amino acid sequence of SEQ ID NO: 1.

The polypeptide of the present invention has at 80% sequence identity with the amino acid sequence of SEQ ID NO: 1, preferably at least 81%, more preferably at least 90% or 95% sequence identity with the amino acid sequence of SEQ ID NO: 1, and preferably has a GalA reductase activity.

In a preferred embodiment, the polypeptide of the present invention is an enzyme with galacturonate (GalA) reductase activity of Aspergillus niger. Within this specification, such polypeptide is also referred to as galacturonate (GalA) reductase of Aspergillus niger.

SEQ ID NO: 1 is the wild-type protein or amino acid sequence of AnGar1, galacturonate (GalA) reductase of Aspergillus niger.

Encoded by gene An16g04770; UniProt Acc. Number A2R7U3

AnGar1 is a protein of 319 amino acids.

```
                10 20 30 40
MGSTSTVADT RFKLNTGAEI PALGLGTWQS GPGEVEKAVA
                50 60 70 80
HAISVGYRHI DTAFAYGNEG EVGKGIKAAI ESGVVKREDL
                90 100 110 120
FVTTKLWSTW HYRVEQALDQ SLKNLGLDYV DLYLVHWPVA
              130 140 150 160
MNPNGNHPNI PTLPDGSRDL HLNHSHINTW KDMEKLVGSG
              170 180 190 200
KTKAIGVCNY SRPYLEELLA QATVVPAVNQ IENHPCLPQQ
              210 220 230 240
EAVDFCKEKG IHITAYSPLG STGSPLLTAE PIVEVAKKKG
              250 260 270 280
VDPATVLLSW HISRGSSVLA KSVNPSRIEG NRNLVALDDA
              290 300 310
DMATIAKYTN DLASKNAFQR FVFPPFKLDF GFPDKIGRV
```

The polypeptides, preferably the GalA reductase variants, according to the invention comprise at least one amino acid substitution at a position corresponding to K261 and/or 8267 of the amino acid sequence of SEQ ID NO: 1 or of an amino acid sequence, which is at least 60% identical, preferably at least 70% identical, more preferably at least 80% identical, even more preferably at least 90% identical, yet more preferably 95% identical, and yet more preferably 99% identical to the amino acid sequence of SEQ ID NO: 1.

In a preferred embodiment, the polypeptides according to the invention comprise at least one amino acid substitution at a position corresponding to K261 and/or 8267 of the amino acid sequence of SEQ ID NO: 1 or of an amino acid sequence, which is at least 80% identical, preferably at least 81% identical, more preferably at least 90% identical, more preferably 95% identical, and yet more preferably 99% identical to the amino acid sequence of SEQ ID NO: 1.

As used herein, the term “at a position corresponding to” means the respective position in SEQ ID No: 1 which, however, in related polypeptide chains can have another relative position number. The equivalent substitution can be determined by comparing a position in both sequences, which may be aligned for the purpose of comparison. The relative position of the amino acid can vary due to different length of the related polypeptide, or deletions or additions of amino acids in the related polypeptide.

The polypeptides according to the invention have a galacturonate (GalA) reductase activity.

As used herein, the term “percent (%) identical” refers to sequence identity between two amino acid sequences. Identity can be determined by comparing a position in both sequences, which may be aligned for the purpose of comparison. When an equivalent position in the compared sequences is occupied by the same amino acid, the molecules are considered to be identical at that position.

Preferably, said amino acid substitution(s) at a position corresponding to K261 and/or R267 of the polypeptides of the present invention leads to or confers
a decreased affinity for NADPH and/or NADP and/or an increased affinity for NADH, and/or
catalytic activity for the reduction of D-galacturonate (GalA) into L-galactonate (GalOA).

In a preferred embodiment, the amino acid substitution at a position corresponding to K261 of the amino acid sequence of SEQ ID NO: 1 is K261M, K261A or K261V, more preferably K261M.

In a preferred embodiment, the amino acid substitution at a position corresponding to R267 of the amino acid sequence of SEQ ID NO: 1 is R267L, R267W, R267F, R267D, R267E, more preferably R267L.

In a preferred embodiment, the polypeptide comprises both amino acid substitutions K261M and R267L.

The present invention preferably provides the following polypeptides/GalA reductase variants:
K261M,
K261A,
K261V,
R267L,
R267W,
R267F,
R267D,
R267E,
K261M/R267L,
K261A/R267L,
K261V/R267L,
K261M/R267W,
K261A/R267W,
K261V/R267W,
K261M/R267F,
K261A/R267F,
K261V/R267F,
K261M/R267D,
K261A/R267D,
K261V/R267D,
K261M/R267E,
K261A/R267E, and/or
K261V/R267E,

More Preferably
K261M,
R267L,
K261M/R267L.

Nucleic Acid Molecules As discussed above, the present invention provides a nucleic acid molecule, coding for a polypeptide according to the present invention.

In one embodiment, the nucleic acid molecule of the present invention further comprises:
vector nucleic acid sequences, preferably expression vector sequences, and/or
promoter nucleic acid sequences and terminator nucleic acid sequences, and/or
comprises other regulatory nucleic acid sequence.

In one embodiment, the nucleic acid molecule of the present invention comprises dsDNA, ssDNA, PNA, CNA, RNA or mRNA or combinations thereof.

The nucleic acid molecules according to the invention preferably comprise nucleic acid sequences, which are (except for the addition of the amino acid substitution(s) according to the invention) identical with the naturally occurring nucleic acid sequence or are codon-optimized for the use in a host cell.

The nucleic acid molecule used according to the present invention is preferably a nucleic acid expression construct.

Nucleic acid expression constructs according to the invention are expression cassettes comprising a nucleic acid molecule according to the invention, or expression vectors comprising a nucleic acid molecule according to the invention or an expression cassette, for example.

A nucleic acid expression construct preferably comprises regulatory sequences, such as promoter and terminator sequences, which are operatively linked with the nucleic acid sequence coding for the polypeptide(s) of the invention.

The nucleic acid expression construct may further comprise 5′ and/or 3′ recognition sequences and/or selection markers.

Host Cells

As discussed above, the present invention provides host cells containing a nucleic acid molecule according to the present invention.

Preferably, the host cells of the present invention express said nucleic acid molecule.

Preferably, a host cell according to the present invention is a fungus cell and more preferably a yeast cell.

The yeast cell is preferably a member of a genus selected from the group of Saccharomyces species, Kluyveromyces sp., Hansenula sp., Pichia sp., Yarrowia sp or Ogataea sp..

The yeast cell is more preferably a member of a species selected from the group of S. cerevisiae, S. bulderi, S. barnetti, S. exiguus, S. uvarum, S. diastaticus, K. lactis, K. marxianus, K. fragilis, H. polymorpha, P. pastoris and Y. lipolytica, such as S. cerevisiae, K. lactis, H. polymorpha, P. pastoris or Y. lipolytica.

In a preferred embodiment, the host cell belongs to the species Saccharomyces cerevisiae.

When the nucleic acid molecule/sequence coding for the polypeptide (preferably GalA reductase variant(s)) of the present invention is expressed in a host cell (preferably a yeast cell), the host cell is imparted the capability to reduce D-galacturonate (GalA) into L-galactonate (GalOA).

In a preferred embodiment, the host cell (preferably yeast cell) of the present invention:
further contains nucleic acid molecules which code for enzymes necessary to provide reducing equivalents from substrates, such as formiate, methanol or polyols, e.g. sorbitol, mannitol, xylitol or glycerol,
   preferably nucleic acid molecules which code for polyol dehydrogenase(s) (DH), such as sorbitol DH, mannitol DH, xylitol DH and glycerol DH, and/or
the nucleic acid molecules which code for alcohol dehydrogenase(s) (ADH) and/or glycerol-phosphate dehydrogenases are deleted.

In one embodiment, the host cell (preferably yeast cell) of the present invention further contain nucleic acid molecules which code for a GalA transporter, such as GatA from Aspergillus niger and/or GAT-1 from Neurospora crassa.

When the nucleic acid molecule/sequence coding for a GalA transporter is expressed in a host cell (preferably a yeast cell), the host cell is imparted the capability to uptake D-galacturonate (GalA).

In one embodiment, the host cell (preferably yeast cell) of the present invention uses glucose and/or other neutral sugar(s), such as galctose, arabinose and xylose, as a co-substrate, i.e. as redox donor. In such embodiment where preferably glucose is used as co-substrate, the nucleic acid molecules which code for alcohol dehydrogenase(s) (ADH) and/or glycerol-phosphate dehydrogenases are preferably deleted in the host cell.

Methods and Uses for Producing GalOA

As discussed above, the present invention provides a method for the production of L-galactonate (GalOA) and/or other bio-based compounds.

Said method comprises the expression of a nucleic acid molecule according to the present invention, preferably in a host cell according to the present invention.

As discussed above, the present invention provides the use of
a polypeptide according to the present invention,
a nucleic acid molecule according to the present invention, or
a host cell according to the present invention,
for the production of L-galactonate (GalOA) and/or other bio-based compounds.

The term “bio-based compounds” or “other bio-based compounds” as used herein refers to chemical compounds and substances, which are obtained from biological materials and raw materials (biomass), particularly by using microorganisms.

The (other) bio-based compounds can be compounds, which are selected from, but not limited to:
galactono-γ-lacton,
vitamin C (ascorbic acid),
ethanol,
isobutanol,
fatty acid(s), and/or
isoprenoid(s).

By utilizing galacturonic acid as sole or additional carbon source (via the conversion to L-galactonate), many different products can be produced, such as ethanol, isobutanol, fatty acid(s), isoprenoid(s).

As discussed above, the present invention provides the use of
a polypeptide according to the present invention,
a nucleic acid molecule according to the present invention, or
a host cell according to the present invention,
for the recombinant fermentation of biomaterial containing D-galacturonate (GalA) and/or other neutral sugar(s), such as galactose, arabinose or xylose.

Said biomaterial containing D-galacturonate (GalA) preferably refers to pectin-containing or pectin-rich biomass, such as pectin-rich agricultural biomass and feedstocks, e.g. sugar beet pulp, citrus fruit peel, agave pulp, grape pomace.

As discussed above, the present invention provides the use of
a polypeptide according to the present invention,
a nucleic acid molecule according to the present invention, or
a host cell according to the present invention,
for the recombinant fermentation of biomaterial containing D-galacturonate (GalA) and glucose.

### Further Description of Preferred Embodiments

Establishing a system for redox-balanced GalA reduction

Previous work by others has shown that, due to its high oxidation state, the reduction of GalA can occur only in the presence of a co-substrate (redox-donor) such as fructose or glucose, but the molar yield of GalOA per mol co-substrate was low (Matsubara et al., 2016). Presumably, the low yields were due to insufficient redox power provided by hexose metabolism. Therefore, we decided to couple the reduction of GalA to the oxidation of the sugar alcohol sorbitol, which exhibits a higher reduction state compared to glucose. Sorbitol must be oxidized to fructose by a sorbitol dehydrogenase (SDH) to enter glycolysis. Thereby, SDH can provide the necessary reducing equivalents in a stoichiometric manner (FIG. 1).

For co-utilization of sorbitol and GalA, different genetic cassettes, each for overexpression of four genes—a sorbitol transporter, an SDH, a GalA transporter and a GalA reductase—were constructed and integrated into the URA3 locus of the hexose-transporter deficient (hxt⁰) strain EBY.VW400020 (Wieczorke et al., 1999). This strain background was chosen to rule out any influence of endogenous hexose transporters on GalA uptake (Protzko et al., 2018). All cassettes contained the endogenous transporter HXT13 for sorbitol uptake (Jordan et al., 2016) and GatA from A. niger (Protzko et al., 2018) for GalA uptake. As GalA reductases, we chose the well-known enzyme TrGar1 and its orthologue from A. niger (AnGar1), for which a GalA reductase activity had not been demonstrated yet. To provide NADPH or NADH, we selected the YlSdr from Yarrowia lipolytica (Napora et al., 2013) or the endogenous Sor (Jordan et al., 2016), respectively. The different combinatorial cassette configurations and resulting strains are listed in Table 3.

To test the concept, we performed shake flask fermentations under aerobic conditions with these strains in sorbitol-containing media with or without GalA supplementation (Figure. 2). In line with previous observations (Jordan et al., 2016), the (NAD⁺-dependent) Sor2 conferred robust growth and sorbitol consumption, both of which were significantly delayed by the addition of GalA. This is likely due to the weak acid toxicity and/or inhibition of sorbitol uptake, as Hxt13 is closely related to the yeast galactose permease Gal2 that was previously reported to be competitively inhibited by GalA (Huisjes et al., 2012). With Sor2, only low concentrations of GalOA (up to 0.68 g L⁻¹) were produced. The behavior of strains expressing the NADP+-dependent YlSdr was the opposite. Without GalA, only marginal growth and a concomitantly slow sorbitol consumption were measured. This can be explained by the accumulation of cytosolic NADPH that, in contrast to NADH, cannot be re-oxidized, leading to a growth arrest (Boles et al., 1993). As expected, the addition of GalA stimulated growth and sorbitol consumption by acting as a redox sink for the accumulating NADPH (see FIG. 1). This is reflected by the substantially higher titer of GalOA in fermentations with YlSdr (up to 2.54 g L⁻¹). For both GalA reductases, TrGar1 and AnGar1, resulting titers and molar yields per mol consumed co-substrate (Y_{P/S}) were similar. Despite slower growth and sorbitol consumption, these yields were about twelvefold higher with NADPH- than NADH-dependent SDH. Altogether, these results show that the supply of suitable reducing equivalents is the most critical engineering target for GalOA production. Moreover, we demonstrate, for the first time, the activity of AnGar1 as a GalA reductase.
Engineering GalA Reductases for NADH-Specificity

To identify the amino acid residues responsible for NADPH binding, we generated the structural models of TrGar1 and AnGar1. The crystal structure of the NADPH-dependent aldehyde reductase AKR1A1 from Sus scrofa (PDB ID 1HQT) was the template; this enzyme shares 37% sequence identity with TrGar1 and AnGar1. The homology models of TrGar1 and AnGar1 were constructed in Molecular Operating Environment (MOE; Chemical Computing Group, https://www.chemcomp.com/). Given the high sequence homology between TrGar1 and AnGar1 (63% identity and 81% similarity), their models are quite similar (FIG. 3A). In the active site, the phosphoryl group of NADPH interacts electrostatically with the two positively charged residues Lys (254 in TrGar1 or 261 in AnGar1) and Arg (260 in TrGar1 or 267 in AnGar1) (FIG. 3B). In MOE, we performed mutation scanning of these residues and examined the protein stability as well as NADH vs. NADPH ligand affinity for the various substitutions (data not shown) and determined protein stability results. We found, for example, that amino acid substitutions with Met (for Lys) and Leu (for Arg) preserve the space occupied by Lys and Arg, respectively, in the NADP binding site well. We determined that the amino acid substitutions K254M and R260L in TrGar1 or the corresponding mutations in AnGar1 (K261M and R267L, respectively), alone or in combination, will result in the desired change of cofactor-specificity (FIG. 3C).

The ability of GalA reductase variants to utilize different cofactors was tested using the sorbitol co-fermentation system as described above, with the exception that enzymes were expressed from multicopy (2 μ) plasmids. AnGaaA, a phylogenetically non-related GalA reductase, which naturally accepts NADPH and, albeit to a lesser extent, NADH (Martens-Uzunova et al., 2008) was included for comparison.

As expected, the two wildtype GalA reductases could only produce a significant amount of GalOA when in combination with YlSdr, i.e., with NADPH as a cofactor (FIG. 4). The lower activity of AnGaaA compared to TrGar1 is consistent with previous observations (Protzko et al., 2018), whereas wildtype TrGar1 and AnGar1 produce comparable amount of GalOA. The mutated variants of TrGar1 showed a decreased activity with NADPH but no considerable activity with NADH. In contrast, both single amino acid substitutions K261M and R267L conferred AnGar1 the ability to accept NADH in addition to NADPH. The double mutant K261M/R267L even imparts NADH-specificity to AnGar1, since significant amounts of GalOA are only produced in combination with Sor2. Importantly, the mutated AnGar1 variants are superior to AnGaaA with NADH, demonstrating the feasibility of the enzyme engineering approach.

To investigate the molecular basis of the above in vivo observations, we furthermore performed in vitro enzyme assays of AnGar1 variants. For this, we selected the wildtype enzyme, the single mutant producing the higher GalOA amount in the NADH background (R267L) and the double mutant K261M/R267L. Consistent with in vivo results, the single mutation only partly and the double mutation almost fully abolished the AnGar1 activity with NADPH as the cofactor (FIG. 5A). When the assay was performed with NADH (600 all three variants showed comparable activities, which were by an order of magnitude lower compared to those with NADPH of the wildtype enzyme. This demonstrates that (also wildtype) AnGar1 accepts NADH, albeit with a substantially lower preference compared to NADPH. However, when NADP was added to competitively inhibit NADH binding, only the mutants were able to reduce GalA using NADH (FIG. 5 B). Whereas the double mutant was fully insensitive to NADP at both NADH concentrations tested, the single mutant showed a considerable sensitivity at the lower (160 μM) but not at the higher (800 μM) NADH concentration. This is consistent with its ability to use NADPH as a cofactor, in contrast to the double mutant (FIGS. 4, 5A).

Together, these data demonstrate that the change of the cofactor preference is due to a dramatically decreased affinity towards NADP(H), which is in accordance with the expectations based on the structure model. In the cellular context, the wildtype enzyme cannot use NADH supplied by Sor2 due to the presence of NADP (which accumulates in the absence of the enzyme that can re-reduce it, i.e. in the absence of YlSdr). Conversely, the mutated variants can bind NADH, since they are not sensitive to the presence of NADP. This notion also explains why AnGar1 mutants produce more GalOA in vivo in the Sor2 background compared to AnGaaA, which also accepts NADH, but with a by an order of magnitude lower affinity (reflected by the Kₘ value) compared to NADPH (Martens-Uzunova et al., 2008).
Co Fermentation of GalA and Glucose Using Engineered GalA Reductases

Sorbitol and GalA co-occur in fruits, but some major sources of pectin, such as sugar beet pulp, do not contain a large amount of the sugar alcohol. Instead, this feedstock contains glucose, galactose, and arabinose as major carbohydrates (Schafer et al., 2020). All these substrates are funneled into glycolysis, which produces NADH in the GAPDH reaction. In S. cerevisiae, the predominant fraction of NADH is re-oxidized by the production of ethanol even under aerobic conditions in the presence of glucose due to the Crabtree effect (Piskur et al., 2006). Thus, glycolysis is redox-neutral, and “superfluous” NADH, resulting from the production of biomass, is re-oxidized through the production of glycerol (Bakker et al., 2001).

To minimize the re-oxidation of NADH, we decided to test the performance of our engineered GalA reductase variants in the strain JWY019, which lacks the main alcohol dehydrogenase ADH1 and the glycerol-phosphate-dehydrogenase genes GPD1 and GPD2 (Wess et al., 2019). In this strain background, the engineered enzymes indeed achieve higher GalOA titers (FIG. 6A), and molar yields per mol consumed glucose (FIG. 6B).

With the K261M/R267L mutant, the yields were more than doubled compared to those observed with the wildtype enzyme. However, they are still below those obtained on sorbitol. This is obviously due to the fact that, despite the deletion of ADH1, GPD1 and GPD2, a significant proportion of NADH is re-oxidized in JWY019 through the residual formation of ethanol (FIG. 6C) and glycerol (FIG. 6D). Strikingly, the production of glycerol is inversely correlated with the increased production of GalOA, supporting the conclusion that GalA acts as a redox sink for the glycolytic NADH.

Conclusion

The present invention for the first time discloses GalA reductases which exhibit a higher conversion rate of GalA into GalOA with NADH compared to NADPH. This allows coupling the GalA reduction with the metabolic pathways of yeast as well as further biotechnological relevant organisms.

The NADH-dependent GalA reductases of the invention can also be used with other co-substrates whose metabolism provides surplus reducing equivalents such as formiate, methanol, mannitol, glycerol or xylitol.

The NADH-dependent GalA reductases of the invention can also be used independently from the use of such co-substrates whose metabolism provides surplus reducing equivalents. The required cofactors can be provided by glycolysis, by eliminating the NADH-consuming reactions, such as synthesis of ethanol (FIG. 8) and/or glycerol when co-substrates such as glucose, galactose, fructose, maltose, arabinose or xylose are used.

Furthermore, we demonstrate herein that the supply of suitable redox cofactors is a critical engineering target to enable efficient reduction of GalA in S. cerevisiae. This can be achieved by feeding a co-substrate exhibiting a high reduction state, such as sorbitol (or other polyols). We show for the first time that AnGar1 has a GalA reductase activity. Using the sorbitol-based screening system and structure-guided mutagenesis, we developed AnGar1 variants that, to our knowledge, represent the first reported GalA reductases with a higher preference for NADH compared to NADPH. The altered cofactor-specificity enables the coupling of GalA reduction to glycolysis, resulting in higher yields of GalOA when glucose is used as a redox donor. Hence, the engineered AnGar1 prove valuable for GalA utilization in pectin-rich hydrolysates, which contain neutral sugars such as glucose, galactose, or arabinose, all of which are funneled into glycolysis. Moreover, the NADH-dependent GalA reductases could facilitate the coupling of GalOA production to the oxidation of glycerol, an abundant waste product that could be supplemented to pectin-rich hydrolysates.

The following examples and drawings illustrate the present invention without, however, limiting the same thereto.

### BRIEF DESCRIPTION OF THE DRAWINGS

FIG. 1. Redox-balanced system for GalOA production.

The cofactors necessary for the reduction of D-galacturonate by GalA reductases (TrGar1, AnGar1 or AnGaaA) to GalOA can be derived from the oxidation of sorbitol by sorbitol dehydrogenases (SDH). By choosing the suitable SDH, either NADH (Sort) or NADPH (YlSdr) can be accumulated. Fructose produced by SDH subsequently enters glycolysis.

FIG. 2. Co Fermentation of GalA and Sorbitol.

Yeast strains expressing indicated enzyme combinations were cultivated in shake flasks in phosphate-buffered SC-media with sorbitol as carbon source either without (dashed lines) or with (solid lines) GalA. Cell growth was monitored photometrically (OD600). Concentrations of sorbitol, GalA, and GalOA were measured by HPLC. Mean values and standard deviations of biological triplicates are shown. Error bars may be smaller than the symbols. Molar yields were calculated as mol GalOA produced per mol of sorbitol consumed after 8 days of cultivation. The same symbols are applied in all panels.

FIG. 3. Structural Homology Models of TrGar1 and AnGar1.

The homology structural models of TrGar1 and AnGar1 were based on the crystal structure of the NADPH-dependent aldehyde reductase AKR1A1 (PDB ID 1HQT).

(A) Overlay of the structures of TrGar1 and AnGar1 showing the binding site of NADPH.

(B) and (C) Close-up of the binding site for the phosphoryl moiety of NADPH in the wild-type (B) vs. the double mutant (C) enzymes (TrGar1 K254M/R260L or ArGar1 K261M/R267L). The double mutant lacks the electrostatic interactions of the NADPH phosphoryl group with Lys (254 in TrGar1 or 261 in ArGar1) and Arg (260 in TrGar1 or in ArGar1) residues.

FIG. 4. Cofactor Dependency of the Reduction of GalA to GalOA in the GAR Enzyme Variants of the Present Invention.

Indicated GalA reductase variants were overexpressed from multicopy plasmids in the strains SiHY007 (YlSdr) and SiHY008 (Sor2) together with NADPH- or NADH-dependent SDH YlSdr or Sor2, respectively. The conversion of GalA into GalOA was measured in culture supernatants of shake flasks after 7 days of cultivation by HPLC analysis. AnGaaA, which naturally accepts NADPH and also NADH, was included for comparison. Mean values and standard deviations of biological triplicates are shown.

FIG. 5. Enzyme Activity Assay of AnGar1 Variants.

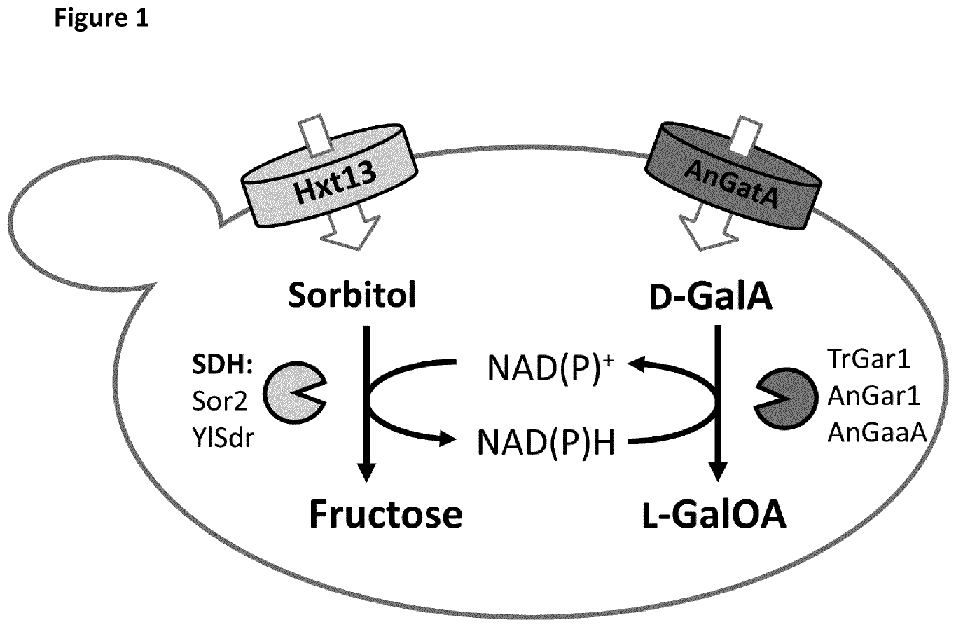

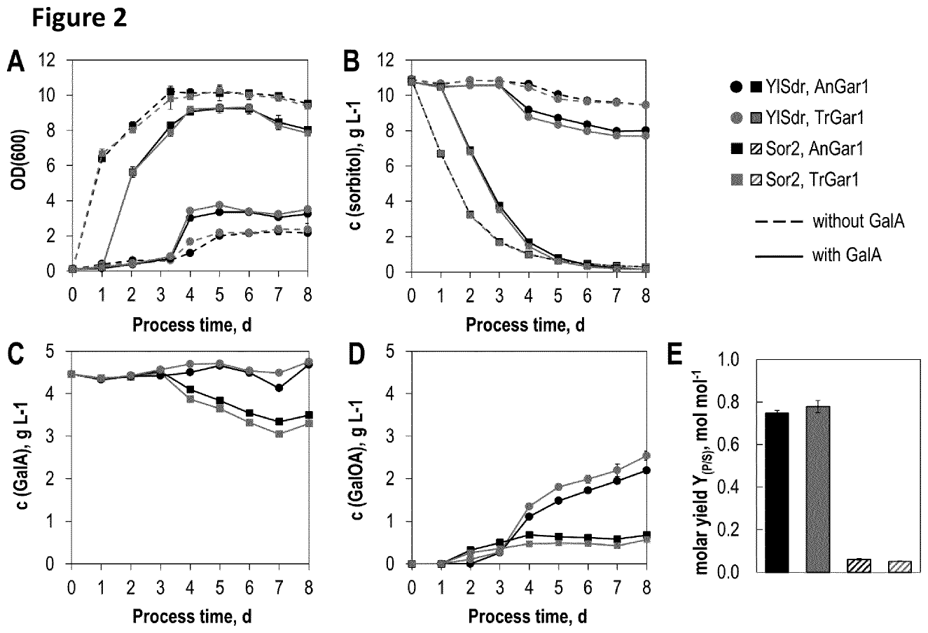

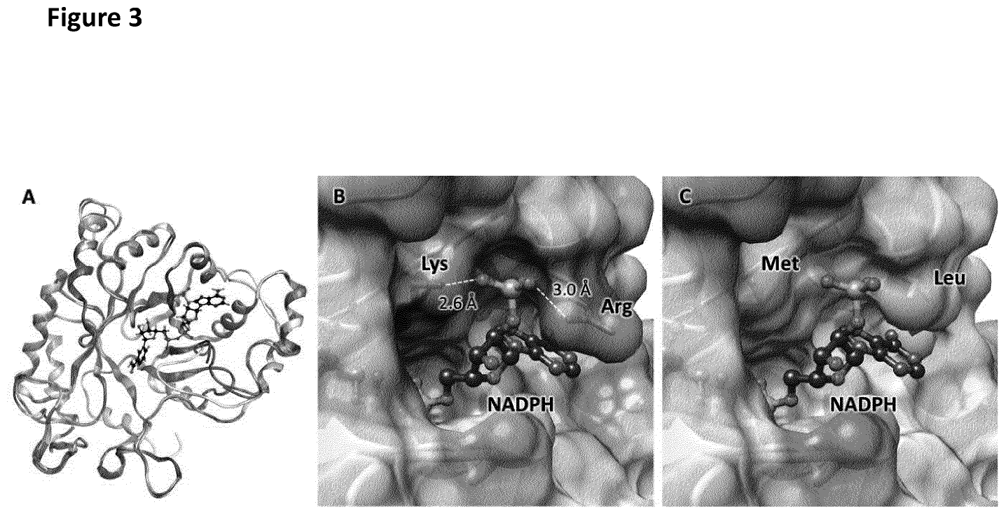

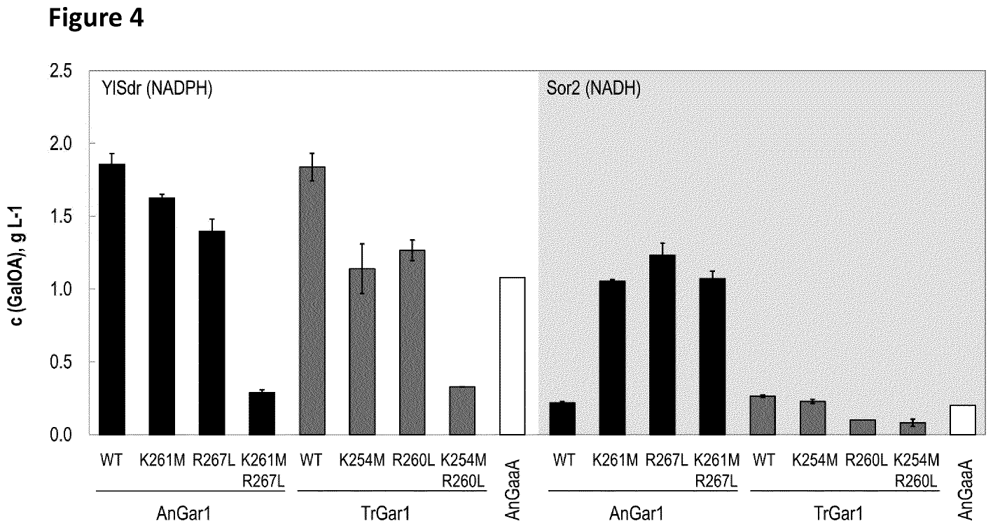

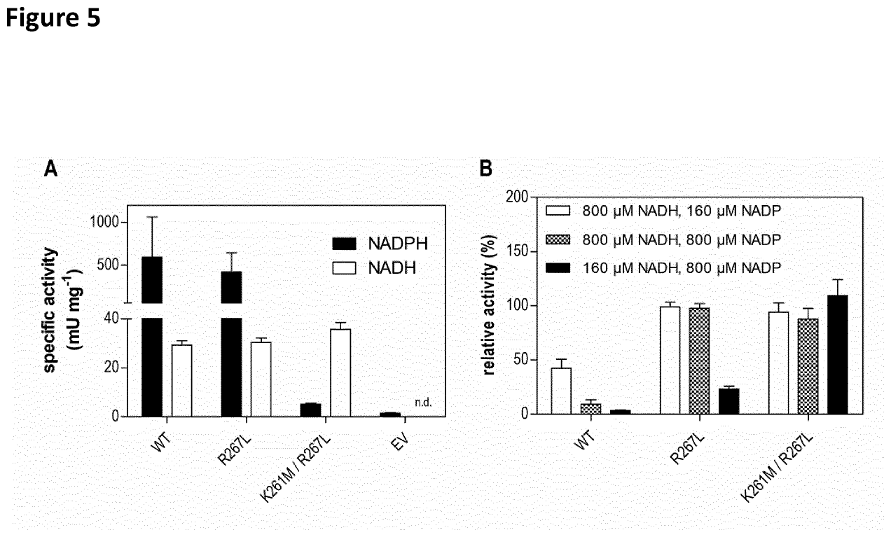

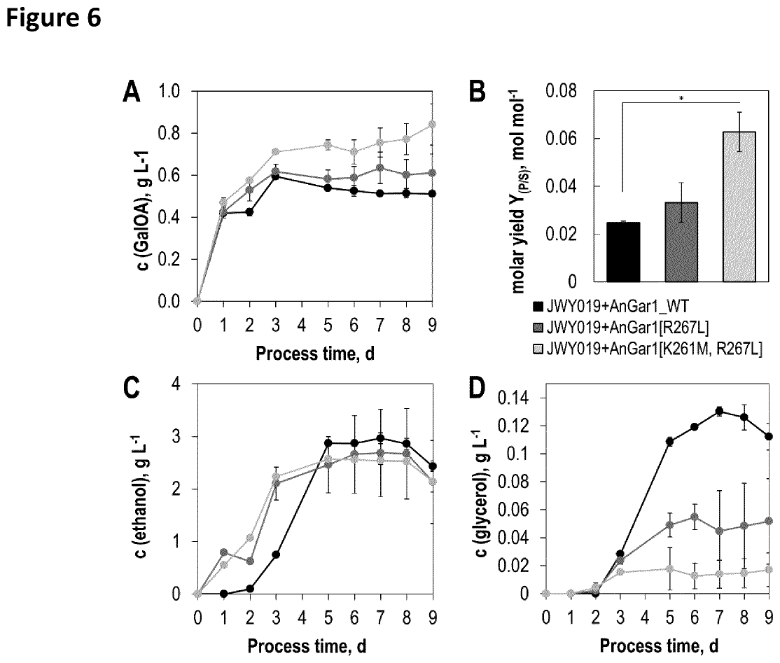

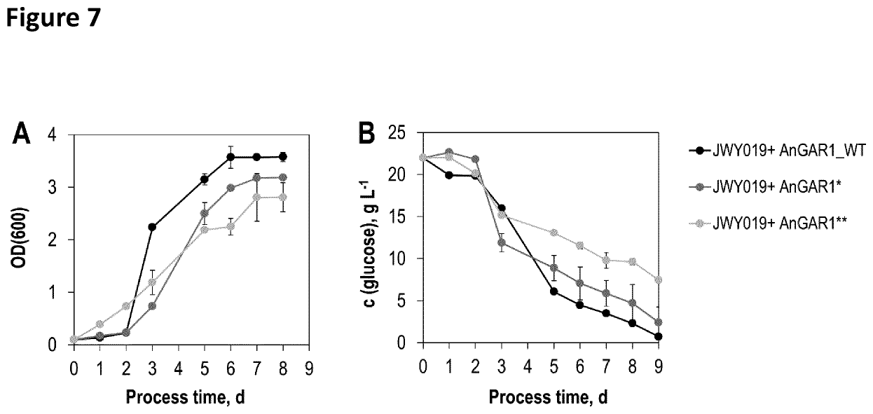

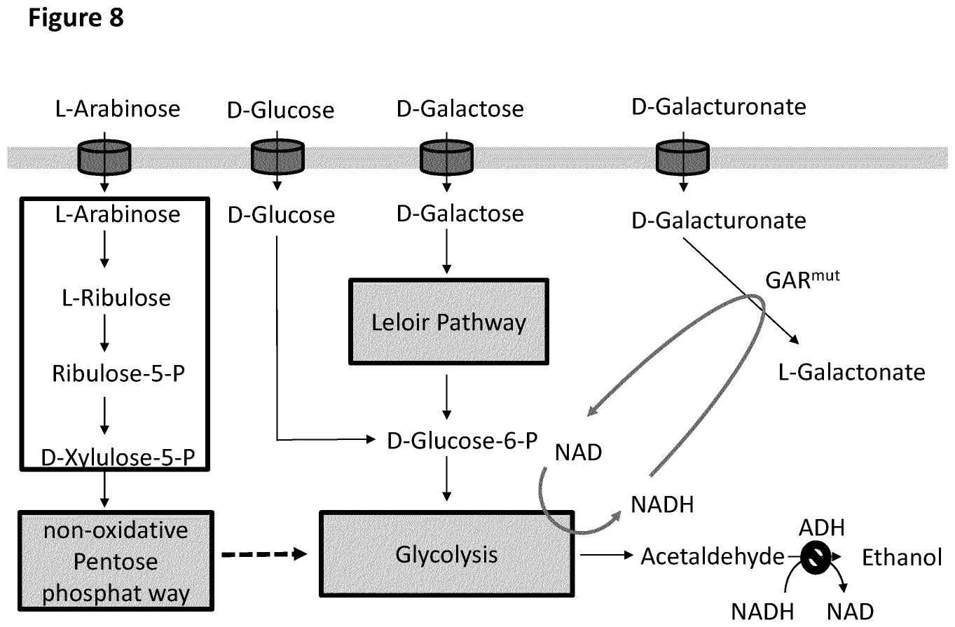

The enzymes were expressed from plasmids in CEN.PK2-1C cells. The cells transformed with the empty vector (EV) were used as a negative control. In (A), the assays were performed with NADPH or NADH alone. The specific activity (mili Units per mg protein, mU mg⁻¹) is shown. The Y axis is divided in two segments to better visualize the lower activities. In (B), the assays were performed with NADH and NADP (oxidized form) as a competitive inhibitor at indicated concentrations. Shown are relative activities, calculated as percent of the activity measured at the respective NADH concentration in the absence of NADP. Error bars represent standard deviation of technical triplicates. n.d., not detectable.

FIG. 6. Performance of Engineered GalA Reductase Variants in Co Fermentations of GalA and Glucose.

Different GalA reductase variants (AnGar1_WT, AnGar1 [R267L] and AnGar1[K261M/R267L] were integrated into the genome of the adh1Δ gpd1Δ gpd2Δ strain JWY019, yielding strains SiHY072, SiHY062 and SiHY063, respectively. The production of GalOA (A, B), ethanol (C) and glycerol (D) were measured in culture supernatants by HPLC analysis. In (B) the molar yields of GalOA (mol per mol consumed glucose) were calculated after 9 days of cultivation. The difference between the wildtype and the double mutant is statistically significant (t-test P<0.005), whereas the difference between the wildtype and the single mutant is not (P>0.05). The corresponding glucose consumption and growth curves are shown in FIG. 7. Mean values and standard deviations of biological triplicates are shown.

FIG. 7. Glucose Consumption and Growth of Strains Expressing GalA Reductase Variants

Different GalA reductase variants (AnGar1_WT, AnGar1[R267L] and AnGar1[K261M/R267L] were integrated into the genome of the adh1Δ gpd1Δ gpd2Δ strain JWY019, yielding strains SiHY072, SiHY062 and SiHY063, respectively. The OD600 (A) and the glucose concentration in the supernatant (B) were monitored over the cultivation time of 9 days via photometry and HPLC analysis, respectively. Mean values and standard deviations of biological triplicates are shown.

FIG. 8. Coupling of GalA Reduction with the Metabolism of Different Sugars.

Shown are the relevant steps of the metabolism of different sugars, which are present in pectin-containing biomass. They all converge in glycolysis which produces NADH. In S. cerevisiae, NADH is re-oxidized mainly via the synthesis of ethanol or glycerol (the latter not shown).

By deleting the alcohol dehydrogenases (ADH) and/or glycerol-phosphate dehydrogenases (the latter not shown) and by using mutated GalA reductases (GAR^{mut}), the fermentative metabolism can be replaced with the reduction of GalA into GalOA.

### EXAMPLES

### Example 1

1. Materials and Methods

1.1 Construction of Expression Cassettes and Strains

The Saccharomyces cerevisiae endogenous open reading frames (ORFs) of HXT13 (YEL069C) and SOR2 (YDL246C) were PCR amplified using the primer pairs SiHP011-SiHP012 (HXT13) and SiHP015-SiHP016 (SOR2). The open reading frame encoding YlSdr (Napora et al., 2013; UniProtKB-Q6CEE9) was amplified from Yarrowia lipolytica genomic DNA using the primer pair SiHP015-SiHP016 (primers are listed in Table 1).

Synthetic ORFs encoding the D-galacturonic acid reductases AnGaaA (Martens-Uzunova and Schaap, 2008; UniProtKB-A8DRH9), TrGar1 (Kuorelahti et al., 2005; UniProtKB-Q3ZFI7) and AnGar1 (UniProtKB-A2R7U3) as well as the D-galacturonic acid transporter AnGatA (Protzko et al., 2018; UniProtKB-A2R3H2) were codon optimized for expression in S. cerevisiae using the online tool JCat (http://www.jcat.de; Grote et al., 2005) and chemically synthesized at Thermo Fisher Scientific. The synthesized ORFs were cloned into pYTK001, the entry plasmid of the Golden Gate Cloning toolkit according to the published protocol (Lee et al., 2015). The resulting plasmids, denoted as pGG3.x, are listed in Table 2.

Site directed mutagenesis for amino acid substitutions was performed on pGG3.6 (AnGAR1) and pGG3.7 (TrGAR1) using the primers listed in Table 1. From the pGG3.x entry plasmids, integrative (SIEV046 and −47, Table 2) or episomal (SiHV057-102, Table 2) expression constructs were generated by combining the ORFs with modules of the Golden Gate toolkit as listed in Table 2 according to the published procedure (Lee et al., 2015).

Novel strains, SiHY001, SiHY002, SiHY003, SiHY004, SiHY007 and SiHY008 (Table 3), were constructed based on the parental strain EBY.VW4000 (Wieczorke et al., 1999) by integrating expression cassettes from SiHV040, SiHV041, SiHV042, SiHV043, SiHV046 and SiHV047, which were digested with NotI before. The strains SiHY062, SiHY063, and SiHY072, which are based on the parental strain JWY019 (Wess et al., 2019), were constructed via integration of the expression cassettes from plasmids SiHV136, SiHV137, and SiHV158 after NotI-digest. The cassettes were integrated into the URA3 locus. Positive transformants were selected on G418 and PCR-verified.

For testing the cofactor-dependent activity of different D-galacturonic acid reductase variants, the appropriate plasmids (SiHV057-102, Table 2) were transformed into SiHY007 and SiHY008.

TABLE 1
Primer list

| | | | SEQ |
|---|---|---|---|
| Primer | | | ID |
| name | Target | Sequence | NO. |
| SiHP011 | Hxt13_fw | CGTCTCGTCGGTCTCATATGTCTAGTGCGCAATCCTCTA | 2 |
| SiHP012 | Hxt13_rev | CGTCTCAGGTCGGTCTCAGGATTCAATCAGAATTCTTTGAG | 3 |
| | | AACTTC | |
| SiHP013 | YlSdr_fw | CGTCTCGTCGGTCTCATATGCCTGCACCAGCAAC | 4 |
| SiHP014 | YlSdr_rev | CGTCTCAGGTCGGTCTCAGGATTCAAGGACAACAGTAGCCG | 5 |
| | | C | |
| SiHP015 | Sor2_fw | CGTCTCGTCGGTCTCATATGTCTCAAAATAGTAACCCTGCA | 6 |
| | | GT | |
| SiHP016 | Sor2_rev | CGTCTCAGGTCGGTCTCAGGATTCATTCAGGACCAAAGATA | 7 |
| | | ATAGTCTT | |
| SiHP046 | TrGar1: K254M_fw | GGTTCTACTGTTTTGGCTATGTCTGTTACTCCAGCT | 8 |
| SiHP047 | TrGar1: K254M_rev | GCTGGAGTAACAGACATAGCCAAAACAGTAGAACCTCTG | 9 |
| SiHP048 | TrGar1: R260L_fw | TACTCCAGCTCTGATCAAGGCTAACTTGGAAATCGTTGACT | 10 |
| | | TGGA | |
| SiHP049 | TrGar1: R260L_rev | AGTTAGCCTTGATCAGAGCTGGAGTAACAGACTTAGCCAAA | 11 |
| | | ACAGTAGAACC | |
| SiHP050 | TrGar1: K254M, | AGTTAGCCTTGATCAGAGCTGGAGTAACAGACATAGCCAAA | 12 |
| | R260L_rev | ACAGTAGAACCTCTGTTAACGTGG | |
| SiHP051 | AnGar1: K261M_fw | CTGTTTTGGCTATGTCTGTTAACCCATCTAGAATCGAAGG | 13 |
| SiHP052 | AnGar1: | GATGGGTTAACAGACATAGCCAAAACAGAAGAACCTCTAGA | 14 |
| | K261M_rev | GATGTGC | |
| SiHP053 | AnGar1: R267L_fw | GTCTGTTAACCCATCTCTGATCGAAGGTAACAGAAACTTGG | 15 |
| | | TTGCTTTGG | |
| SiHP054 | AnGar1: R267L_rev | CCAAGTTTCTGTTACCTTCGATCAGAGATGGGTTAACAGAC | 16 |
| | | TTAGCCAAAACAGAAGAAC | |
| SiHP055 | AnGar1: K261M, | CCAAGTTTCTGTTACCTTCGATCAGAGATGGGTTAACAGAC | 17 |
| | R267L_rev | ATAGCCAAAACAGAAGAACCTCTAGAGATGTGC | |

TABLE 2
Plasmid list

| Plasmid name | Content | Source |
|---|---|---|
| pGG3.2 | pYTK001-HXT13 | this study |
| pGG3.3 | pYTK001-YlSDR | this study |
| pGG3.4 | pYTK001-SOR2 | this study |
| pGG3.6 | pYTK001-AnGAR1 | this study |
| pGG3.6 | pYTK001-AnGAR1 [K261M] | this study |
| K261M | | |
| pGG3.6 R267L | pYTK001-AnGAR1 [R267L] | this study |
| pGG3.6 | pYTK001-AnGAR1 [K261M, R267L] | this study |
| K261M, R267L | | |
| pGG3.7 | pYTK001-TrGAR1 | this study |
| pGG3.7 | pYTK001-TrGAR1 [K254M] | this study |
| K254M | | |
| pGG3.7 R260L | pYTK001-TrGAR1 [R260L] | this study |
| pGG3.7 | pYTK001-TrGAR1 [K254M, R260L] | this study |
| K254M, R260L | | |
| pGG3.9 | pYTK001-AnGATA | this study |
| pGG3.18 | pYTK001-AnGAAA | this study |
| SiHV005 | Empty expression backbone pURA3-URA3-tURA3, 2μ, KanR- | this study |
| | ColE1 | |
| SiHV033 | -URA3 5′Hom-ConLS′-GFP-dropout-ConRE′-KanMX-URA | this study |
| | 3′Hom-KanR-ColE1 URA3 integration plasmid, with KanMX | |
| | dominant marker | |
| SiHV040 | URA3_5′-pCCW12-AnGATA-tPGK1-pPGK1-AnGAR1-tENO1- | this study |
| | pTDH3-HXT13-tSSA1-pTEF2-YlSDR-tADH1- pAgTEF-KanMX- | |
| | tAgTEF-URA3_3′- KanR-ColE1 | |
| SiHV041 | URA3_5′-pCCW12-AnGATA-tPGK1-pPGK1-TrGAR1-tENO1- | this study |
| | pTDH3-HXT13-tSSA1-pTEF2-YlSDR-tADH1- pAgTEF-KanMX- | |
| | tAgTEF-URA3_3′- KanR-ColE1 | |
| SiHV042 | URA3_5′-pCCW12-AnGATA-tPGK1-pPGK1-AnGAR1-tENO1- | this study |
| | pTDH3-HXT13-tSSA1-pTEF2-SOR2-tADH1- pAgTEF-KanMX- | |
| | tAgTEF-URA3_3′- KanR-ColE1 | |
| SiHV043 | URA3_5′-pCCW12-AnGATA-tPGK1-pPGK1-TrGAR1-tENO1- | this study |
| | pTDH3-HXT13-tSSA1-pTEF2-SOR2-tADH1- pAgTEF-KanMX- | |
| | tAgTEF-URA3_3′- KanR-ColE1 | |
| SiHV046 | URA3_5′-pCCW12-AnGATA-tPGK1-pTDH3-HXT13-tSSA1- | this study |
| | pTEF2-YlSDR-tADH1-pAgTEF-KanMX-tAgTEF-URA3_3′- KanR- | |
| | ColE1 | |
| SiHV047 | URA3_5′-pCCW12-AnGATA-tPGK1-pTDH3-HXT13-tSSA1- | this study |
| | pTEF2-SOR2-tADH1-pAgTEF-KanMX-tAgTEF-URA3_3'- KanR- | |
| | ColEl | |
| SiHV057 | pPGK1-AnGAAA-tENO1, pURA3-URA3-tURA3, 2μ, KanR-ColE1 | this study |
| SiHV058 | pPGK1-TrGAR1 [K254M]-tENO1, pURA3-URA3-tURA3, 2μ, | this study |
| | KanR-ColE1 | |
| SiHV059 | pPGK1-TrGAR1 [R260L]-tENO1, pURA3-URA3-tURA3, 2μ, | this study |
| | KanR-ColE1 | |
| SiHV060 | pPGK1-TrGAR1 [K254M, R260L]-tENO1, pURA3-URA3-tURA3, | this study |
| | 2μ, KanR-ColE1 | |
| SiHV074 | pPGK1-TrGAR1-tENO1, pURA3-URA3-tURA3, 2μ, KanR-ColE1 | this study |
| SiHV079 | pPGK1-AnGAR1-tENO1, pURA3-URA3-tURA3, 2μ, KanR-ColE1 | this study |
| SiHV100 | pPGK1-AnGAR1 [K261M]-tENO1, pURA3-URA3-tURA3, 2μ, | this study |
| | KanR-ColE1 | |
| SiHV101 | pPGK1-AnGAR1 [R267L]-tENO1, pURA3-URA3-tURA3, 2μ, | this study |
| | KanR-ColE1 | |
| SiHV102 | pPGK1-AnGAR1 [K261M, R267L]-tENO1, pURA3-URA3-tURA3, | this study |
| | 2μ, KanR-ColE1 | |
| SiHV115 | pTEF1-AnGAR1 [K261M, R267L]-tTDH1, URA3, 2μ, KanR- | this study |
| | ColE1 | |
| SiHV127 | pTEF1-AnGAR1 [R267L]-tTDH1, URA3, 2μ, KanR-ColE1 | this study |
| SiHV128 | URA3_5′-pCCW12-AnGATA-tPGK1-pPGK1-AnGAR1 [K261M]- | this study |
| | tENO1-pTDH3-HXT13-tSSA1-pTEF2-SOR2-tADH1- pAgTEF- | |
| | KanMX-tAgTEF-URA3_3′- KanR-ColE1 | |
| SiHV129 | URA3_5′-pCCW12-AnGATA-tPGK1-pPGK1-AnGAR1 [K261M, | this study |
| | R267L]-tENO1-pTDH3-HXT13-tSSA1-pTEF2-SOR2-tADH1- | |
| | pAgTEF-KanMX-tAgTEF-URA3_3′- KanR-ColE1 | |
| SiHV136 | URA3_5′-pCCW12-AnGATA-tPGK1-pPGK1-AnGAR1[R267L]- | this study |
| | tENO1- pAgTEF-KanMX-tAgTEF-URA3_3′- KanR-ColE1 | |
| SiHV137 | URA3_5′-pCCW12-AnGATA-tPGK1-pPGK1-AnGAR1 [R267L, | this study |
| | K261M]-tENO1- pAgTEF-KanMX-tAgTEF-URA3_3'- KanR- | |
| | ColE1 | |
| SiHV158 | URA3_5′-pCCW12-AnGATA-tPGK1-pPGK1-AnGAR1-tENO1- | this study |
| | pAgTEF-KanMX-tAgTEF-URA3_3′- KanR-ColE1 | |

TABLE 3
Strain list
For the genotypes, the standard nomenclature is used. Under “relevant genotype” the parental strains
are indicated in bold. The open reading frames relevant for GalA utilization are underlined. The
prefixes “p” and “t” denote promoters and terminators, respectively.

| Strain name | Relevant Genotype | Source |
|---|---|---|
| CEN.PK2-1C | MATa leu2-3, 112 ura3-52 trp1-289 his3-1 MAL2-8c SUC2 | EUROSCARF |
| EBY.VW4000 | CEN.PK2-1C Δhxt1-17 Δgal2 Δstl1::loxP Aagt1::loxP | Wieczorke et |
| | Δmph2::loxP Δmph3::loxP | al., 1999 |
| JWY019 | MATa; MAL2-8c; SUC2; Δilv2; Δbdh1; Δbdh2; Δleu4; | Wess et al., |
| | Δleu9; Δecm31; Δilv1; Δadh1; Δgpd1; Δgpd2 | 2019 |
| SiHY001 | EBY.VW4000 Δura3::pCCW12-AnGATA-tPGK1-pPGK1- | this study |
| | AnGAR1-tENO1-pTDH3-HXT13-tSSA1-pTEF2-YlSDR- | |
| | tADH1-pAgTEF-KanMX-tAgTEF; constructed from | |
| | transformation with SiHV040 | |
| SiHY002 | EBY.VW4000 Δura3::pCCW12-AnGATA-tPGK1-pPGK1- | this study |
| | TrGAR1-tENO1-pTDH3-HXT13-tSSA1-pTEF2-YlSDR- | |
| | tADH1- pAgTEF-KanMX-tAgTEF; constructed from | |
| | transformation with SiHV041 | |
| SiHY003 | EBY.VW4000 Δura3::pCCW12-AnGATA-tPGK1-pPGK1- | this study |
| | AnGAR1-tENO1-pTDH3-HXT13-tSSA1-pTEF2-SOR2- | |
| | tADH1-pAgTEF-KanMX-tAgTEF; constructed from | |
| | transformation with SiHV042 | |
| SiHY004 | EBY.VW4000 Δura3::pCCW12-AnGATA-tPGK1-pPGK1- | this study |
| | TrGAR1-tENO1-pTDH3-HXT13-tSSA1-pTEF2-SOR2- | |
| | tADH1- pAgTEF-KanMX-tAgTEF; constructed from | |
| | transformation with SiHV043 | |
| SiHY007 | EBY.VW4000 Δura3::pCCW12-AnGATA-tPGK1-pTDH3- | this study |
| | HXT13-tSSA1-pTEF2-YlSDR-tADH1-pAgTEF-KanMX- | |
| | tAgTEF; constructed from transformation with SiHV046 | |
| SiHY008 | EBY.VW4000 Δura3::pCCW12-AnGATA-tPGK1-pTDH3- | this study |
| | HXT13-tSSA1-pTEF2-SOR2-tADH1-pAgTEF-KanMX- | |
| | tAgTEF; constructed from transformation with SiHV047 | |
| SiHY030 | CEN.PK2-1C Δura3::pCCW12-AnGATA -pPGK1- | this study |
| | AnGAR1 [R267L]-pTDH3-HXT13-pTEF2-SOR2-KanMX- | |
| | tAgTEF; constructed from transformation with SiHV129 | |
| SiHY032 | CEN.PK2-1C Δura3::pCCW12-AnGATA -pPGK1- | this study |
| | AnGAR1 [K261M, R267L]-pTDH3-HXT13-pTEF2-SOR2- | |
| | KanMX-tAgTEF; constructed from transformation with | |
| | SiHV128 | |
| SiHY062 | JWY019 Δura3::pCCW12-AnGATA-tPGK1-pPGK1- | this study |
| | AnGAR1 [R267L]-pAgTEF-KanMX-tAgTEF; constructed | |
| | from transformation with SiHV136 | |
| SiHY063 | JWY019 Δura3::pCCW12-AnGATA-tPGK1-pPGK1- | this study |
| | AnGAR1 [R267L, K261M]-pAgTEF-KanMX-tAgTEF; | |
| | constructed from transformation with SiHV137 | |
| SiHY072 | JWY019 Δura3::pCCW12-AnGATA-tPGK1-pPGK1- | this study |
| | AnGAR1-pAgTEF-KanMX-tAgTEF; constructed from | |
| | transformation with SiHV158 | |

1.2 Transformation of Yeast Cells

In general, for yeast cell transformation 50 mL YPD culture was inoculated with 1 mL of an YPD preculture and agitated at 200 rpm and 30° C. The optical density was measured at 600 nm wave-length. When OD₆₀₀=0.8−1 was reached, the culture was pelleted at 3000×g for 3 min and washed with 25 mL sterile water. Cells equivalent to 5 OD₆₀₀ units were pelleted at 5000×g for 1 min and used for one transformation. To this end, 240 μL 50% (w/w) polyethylenglycol, 36 μL 1 M lithium acetate, 10 μL ssDNA and either 250 ng of plasmid-based or 5000 ng of linear DNA in 64 μL water were added to the cells. The reaction set-up was mixed thoroughly and incubated at 42° C. for 20 min. Subsequently, the cells were pelleted at 5000×g for 30 s, resuspended in 500 μL YPD medium and spread on appropriate plates. Successfully transformed cells were expected to form colonies after 2-4 days of incubation at 30° C.

1.3 Cultivation of Yeast Cells

Colonies of strains transformed with plasmids for expression of different D-galacturonic acid reductase variants were scraped off for an overnight preculture in synthetic complete medium lacking uracil (SC-Ura) supplemented with 2% (w/v) maltose. Precultures of non-plasmid strains were started from a single colony in synthetic complete medium with all essential medium compounds supplemented. The main culture was cultivated in a 300 mL shake flask in 50 mL SC-Ura, supplemented with 0.5% (w/v) D-galacturonic acid and 1% (w/v) sorbitol or 2% (w/v) glucose, respectively, at 30° C. and shaking at 200 rpm. The medium was buffered with 100 mM potassium phosphate, pH 6.3. The growth was monitored through OD₆₀₀-measurement and samples were withdrawn for HPLC-analysis.

1.4 HPLC Analysis

The samples were treated with 5-sulfosalycilic acid to a final concentration of 5% (w/v). Analysis was done using an Ultimate 3000 HPLC system (Thermo Fisher Scientific) equipped with a NucleoGel Sugar 810 H (Macherey and Nagel) column. The column temperature was set to 30° C. and the eluent (5 mM H₂SO₄) flow rate was 0.4 mL/min under isocratic conditions. The signal was recorded using a refractive index detector (Shodex RI-101, Shoko Scientific Co.).

1.5 Protein Extraction and Enzyme Assays

CEN.PK2-1C cells transformed with AnGar1 plasmids (SiHV079, SiHV101 and SiHV102) or with the empty plasmid as a control were grown in 50 ml SC-Ura media containing 2% (w/v) glucose until an OD₆₀₀=2.0-2.5. Subsequently, cells were harvested by centrifugation, washed and stored at −80° C. until further processing. After thawing on ice, the cells were mechanically disrupted in 10 mM potassium phosphate buffer (pH 7.2) by shaking (10 min at 4° C.) with glass beads (0.45 mm diameter) using a Vibrax cell disruptor (Janke & Kunkel, Staufen, Germany) and the cell debris was subsequently removed by centrifugation (15,000×g, 5 min, 4° C.). Protein concentration of clear crude extracts was determined by the Bradford method, using bovine serum albumin as a standard. Enzyme assays were performed basically as described previously (Martens-Uzunova et al., 2008). In detail, the reaction mixtures contained (in 200 μl) 10 mM potassium phosphate buffer (pH 7.2), 100 mM GalA, 160 or 800 μM NADPH or NADH and NADP as a competitive inhibitor, where indicated. The reaction was started by adding 10 μl of the cell lysate. The oxidation of NAD(P)H during 10 min was recorded by measuring the change of the absorbance at 340 nm. The specific activities (expressed as mili Units, mU per mg protein) were calculated by dividing the slope measured at 340 nm by the reaction time and protein amount in the reaction mixture.

1.6 Modeling of AnGar1 and TnGar1.

The homology models of AnGar1 and TrGar1 were generated with the ‘Homology Model’ function of the program package Molecular Operating Environment (MOE; Chemical Computing Group, https://www.chemcomp.com/), using as a template the crystal structure of the NADPH-dependent aldehyde reductase AKR1A1 from Sus scrofa (PDB ID 1HQT). The amino acid sequence identity and similarity between AKR1A1 and AnGar1 (or TnGar1) are 37% and 59%, respectively. The homology models generated were scored with GB/VI. The mutation residue scan and resulting protein stability and ligand affinity parameters were performed in MOE Protein Designing function with the Forcefields Amber10 and EHT.

### REFERENCES

Bakker, B. M. et al. Stoichiometry and compartmentation of NADH metabolism in Saccharomyces cerevisiae. FEMS Microbiol. Rev. 25, 15-37; 10.1111/j.1574-6976.2001.tb00570.x (2001).
Biz A, Sugai-Guerios M R, Kuivanen J, Maaheimo H, Krieger N, Mitchell D A, Richard P. The introduction of the fungal D-galacturonate pathway enables the consumption of D-galacturonic acid by Saccharomyces cerevisiae. Microb Cell Fact 2016 Aug. 18; 15(1):144. PubMed ID 27538689
Boles, E., Lehnert, W. & Zimmermann, F. K. The role of the NAD-dependent glutamate dehydrogenase in restoring growth on glucose of a Saccharomyces cerevisiae phosphoglucose isomerase mutant. Eur. J. Biochem. 217, 469-477; 10.1111/j.1432-1033.1993.tb18266.x (1993)
Grote, A, Hiller, K, Scheer, M, Münch, R, Nörtemann, B, Hempel, DC, Jahn, D. JCat: A novel tool to adapt codon usage of a target gene to its potential expression host. Nucleic Acids Res 2005 Jul. 1; 33, 526-531. PMID: 15980527
Huisjes, E. H., Hulster, E. de, van Dam, J. C., Pronk, J. T. & van Maris, A. J. A. Galacturonic acid inhibits the growth of Saccharomyces cerevisiae on galactose, xylose, and arabinose. Appl. Environ. Microbiol. 78, 5052-5059; 10.1128/AEM.07617-11 (2012).
Jordan P, Choe J Y, Boles E, Oreb M. Hxt13, Hxt15, Hxt16 and Hxt17 from Saccharomyces cerevisiae represent a novel type of polyol transporters. Sci Rep. 2016 Mar. 21; 6:23502. PMID: 26996892
Kuorelahti S, Kalkkinen N, Penttila M, Londesborough J, Richard P. Identification in the mold Hypocrea jecorina of the first fungal D-galacturonic acid reductase. Biochemistry. 2005 Aug. 23; 44(33):11234-40. PMID: 16101307.
Lee, M E, DeLoache, W C, Cervantes, B, Dueber, J E. A Highly Characterized Yeast Toolkit for Modular, Multipart Assembly. ACS Synth Biol 2015 Sep. 18; 4(9):975-86. PMID: 25871405
Martens-Uzunova and Schaap. An evolutionary conserved d-galacturonic acid metabolic pathway operates across filamentous fungi capable of pectin degradation. Fungal Genet Biol. 2008 November; 45(11):1449-57. PMID: 18768163.
Matsubara, T., Hamada, S., Wakabayashi, A. & Kishida, M. Fermentative production of L-galactonate by using recombinant Saccharomyces cerevisiae containing the endogenous galacturonate reductase gene from Cryptococcus diffluens. J. Biosci. Bioeng. 122, 639-644; 10.1016/j.jbiosc.2016.05.002 (2016).
Protzko R J, Latimer L N, Martinho Z, de Reus E, Seibert T, Benz J P, Dueber J E. Engineering Saccharomyces cerevisiae for co-utilization of D-galacturonic acid and D-glucose from citrus peel waste. Nat Commun. 2018 Nov. 29; 9(1):5059. PMID: 30498222.
Napora K, Wrodnigg T M, Kosmus P, Thonhofer M, Robins K, Winkler M. Yarrowia lipolytica dehydrogenase/reductase: an enzyme tolerant for lipophilic compounds and carbohydrate substrates. Bioorg Med Chem Lett. 2013 Jun. 1; 23(11):3393-5. PMID: 23608762
Piskur, J., Rozpedowska, E., Polakova, S., Merico, A. & Compagno, C. How did Saccharomyces evolve to become a good brewer? Trends Genet. 22, 183-186; 10.1016/j.tig.2006.02.002 (2006).
Ryan, 0. W. et al. Selection of chromosomal DNA libraries using a multiplex CRISPR system. eLife 3, 345; 10.7554/eLife.03703 (2014)
Schafer, D., Schmitz, K., Weuster-Botz, D. & Benz, J. P. Comparative evaluation of Aspergillus niger strains for endogenous pectin-depolymerization capacity and suitability for D-galacturonic acid production. Bioproc. Biosyst. Eng. [published online ahead of print, 2020 Apr. 23] 10.1007/s00449-020-02347-z (2020).
Schmitz K, Protzko R, Zhang L, Benz J P. Spotlight on fungal pectin utilization-from phytopathogenicity to molecular recognition and industrial applications. Appl Microbiol Biotechnol. 2019 March; 103(6):2507-2524. PMID: 30694345.
Wess, J., Brinek, M. & Boles, E. Improving isobutanol production with the yeast Saccharomyces cerevisiae by successively blocking competing metabolic pathways as well as ethanol and glycerol formation. Biotechnol. Biofuels 12, 335; 10.1186/s13068-019-1486-8 (2019).
Wieczorke, R, Krampe, S, Weierstall, T, Freidel, K, Hollenberg, CP, Boles, E. Concurrent knock-out of at least 20 transporter genes is required to block uptake of hexoses in Saccharomyces cerevisiae. FEBS Lett 1999 Dec. 31; 464(3):123-8.

1. A polypeptide comprising an amino acid substitution at a position corresponding to K261 and/or R267 of the amino acid sequence of SEQ ID NO: 1, wherein the polypeptide has at least 80% sequence identity with the amino acid sequence of SEQ ID NO: 1.

2. The polypeptide according to claim 1, wherein the polypeptide is an enzyme with galacturonate (GalA) reductase activity of Aspergillus niger.

3. The polypeptide according to claim 1, wherein the amino acid substitution at a position corresponding to K261 of the amino acid sequence of SEQ ID NO: 1 is K261M, K261A or K261V.

4. The polypeptide according to claim 1, wherein the amino acid substitution at a position corresponding to R267 of the amino acid sequence of SEQ ID NO: 1 is R267L, R267W, R267F, R267D, or R267E.

5. The polypeptide according to claim 1, comprising the amino acid substitutions K261M and R267L.

6. The polypeptide according to claim 1, wherein the amino acid substitution at a position corresponding to K261 and/or R267 confers
a decreased affinity for NADPH and/or NADP and/or an increased affinity for NADH, and/or
catalytic activity for the reduction of D-galacturonate (GalA) into L-galactonate (GalOA).

7. A nucleic acid molecule, encoding a polypeptide according to claim 1.

8. The nucleic acid molecule of claim 7, further comprising expression vector sequences, and/or comprising promoter nucleic acid sequences and terminator nucleic acid sequences, and/or comprising other regulatory nucleic acid sequences.

9. The nucleic acid molecule of claim 7, wherein the nucleic acid molecule comprises dsDNA, ssDNA, PNA, CNA, RNA or mRNA or a combination thereof.

10. A host cell, containing a nucleic acid molecule according to claim 7, wherein said host cell is preferably a fungus cell.

11. The host cell according to claim 10, which belongs to the species Saccharomyces cerevisiae.

12. The host cell according to claim 10, further containing nucleic acid molecules that encode for enzymes necessary to provide reducing equivalents from substrates, selected from formiate, methanol and polyols,
and/or wherein nucleic acid molecules that encode for alcohol dehydrogenase(s) (ADH) and/or glycerol-phosphate dehydrogenases are deleted.

13. The host cell according to claim 10, further containing nucleic acid molecules that encode a GalA transporter.

14. The host cell according to claim 13, which can reduce D-galacturonate (GalA) into L-galactonate (GalOA),
and/or which uses glucose and/or other neutral sugar(s) as a co-substrate.

15. A method for the production of L-galactonate (GalOA) and/or one or more other bio-based compounds, comprising expression in a host cell of a nucleic acid molecule according to claim 7,
wherein the one or more other bio-based compounds are selected from
galactono-γ-lacton,
vitamin C (ascorbic acid),
ethanol,
isobutanol,
fatty acid(s), and
isoprenoid(s).

16. (canceled)

17. A method of fermenting a biomaterial containing D-galacturonate (GalA),
and/or a biomaterial containing D-galacturonate (GalA) and glucose and/or other neutral sugar(s) wherein said method comprises expression, in a host cell, of a nucleic acid according to claim 7.

18. The polypeptide according to claim 1, comprising one or both of the following amino acid substitutions: K261M and R267L.

19. The host cell according to claim 7, wherein the cell is a yeast cell.

20. The host cell according to claim 12, containing nucleic acid molecules that encode polyol dehydrogenase(s) (DH).

21. The host cell according to claim 13, wherein the GalA transporter is GatA from Aspergillus niger and/or GAT-1 from Neurospora crassa.


SEQUENCE LISTING (17 sequences)

SEQ ID NO: 1 - protein, 319 aa, Aspergillus niger
MGSTSTVADTRFKLNTGAEIPALGLGTWQSGPGEVEKAVAHAISVGYRHIDTAFAYGNEGEVGKGIKAAIESGVVKREDLFVTTKLWSTWHYRVEQALDQSLKNLGLDYVDLYLVHWPVAMNPNGNHPNIPTLPDGSRDLHLNHSHINTWKDMEKLVGSGKTKAIGVCNYSRPYLEELLAQATVVPAVNQIENHPCLPQQEAVDFCKEKGIHITAYSPLGSTGSPLLTAEPIVEVAKKKGVDPATVLLSWHISRGSSVLAKSVNPSRIEGNRNLVALDDADMATIAKYTNDLASKNAFQRFVFPPFKLDFGFPDKIGRV

SEQ ID NO: 2 - DNA, 39 bp, Artificial Sequence
  Primer
cgtctcgtcggtctcatatgtctagtgcgcaatcctcta

SEQ ID NO: 3 - DNA, 47 bp, Artificial Sequence
  Primer
cgtctcaggtcggtctcaggattcaatcagaattctttgagaacttc

SEQ ID NO: 4 - DNA, 34 bp, Artificial Sequence
  Primer
cgtctcgtcggtctcatatgcctgcaccagcaac

SEQ ID NO: 5 - DNA, 42 bp, Artificial Sequence
  Primer
cgtctcaggtcggtctcaggattcaaggacaacagtagccgc

SEQ ID NO: 6 - DNA, 43 bp, Artificial Sequence
  Primer
cgtctcgtcggtctcatatgtctcaaaatagtaaccctgcagt

SEQ ID NO: 7 - DNA, 49 bp, Artificial Sequence
  Primer
cgtctcaggtcggtctcaggattcattcaggaccaaagataatagtctt

SEQ ID NO: 8 - DNA, 36 bp, Artificial Sequence
  Primer
ggttctactgttttggctatgtctgttactccagct

SEQ ID NO: 9 - DNA, 39 bp, Artificial Sequence
  Primer
gctggagtaacagacatagccaaaacagtagaacctctg

SEQ ID NO: 10 - DNA, 45 bp, Artificial Sequence
  Primer
tactccagctctgatcaaggctaacttggaaatcgttgacttgga

SEQ ID NO: 11 - DNA, 52 bp, Artificial Sequence
  Primer
agttagccttgatcagagctggagtaacagacttagccaaaacagtagaacc

SEQ ID NO: 12 - DNA, 65 bp, Artificial Sequence
  Primer
agttagccttgatcagagctggagtaacagacatagccaaaacagtagaacctctgttaacgtgg

SEQ ID NO: 13 - DNA, 40 bp, Artificial Sequence
  Primer
ctgttttggctatgtctgttaacccatctagaatcgaagg

SEQ ID NO: 14 - DNA, 48 bp, Artificial Sequence
  Primer
gatgggttaacagacatagccaaaacagaagaacctctagagatgtgc

SEQ ID NO: 15 - DNA, 50 bp, Artificial Sequence
  Primer
gtctgttaacccatctctgatcgaaggtaacagaaacttggttgctttgg

SEQ ID NO: 16 - DNA, 60 bp, Artificial Sequence
  Primer
ccaagtttctgttaccttcgatcagagatgggttaacagacttagccaaaacagaagaac

SEQ ID NO: 17 - DNA, 74 bp, Artificial Sequence
  Primer
ccaagtttctgttaccttcgatcagagatgggttaacagacatagccaaaacagaagaacctctagagatgtgc